(19) United States
(12) Patent Application Publication
(10) Pub. No.: US 2009/0087924 A1
(43) Pub. Date: Apr. 2, 2009

(54) MICROFLUIDIC REVERSE AFFINITY-BLOT DEVICE

(76) Inventors: Magdalena Bynum, San Jose, CA (US); Timothy Beerling, San Francisco, CA (US); Brian Peter, Los Altos, CA (US); Marc Valer Serra, San Francisco, CA (US); Hui Wang, Cupertino, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/864,982

(22) Filed: Sep. 29, 2007

(51) Int. Cl. G01N 33/543; B01J 19/00; G01N 21/64

(52) U.S. Cl. 436/518; 422/68.1; 422/82.08; 422/82.09

(57) ABSTRACT

The microfluidic reverse affinity-blot device of the present disclosure combines affinity binding for isolation and/or enrichment of protein(s) from a sample, followed by separation/identification thereof. In general terms, a microfluidic reverse affinity-blot device is a closed system of interconnected components that is comprised of defined points of entry and exit, wherein the interconnected components include a capture region upstream from a protein separation region and subsequent detection region. Methods of use are also described.

### BACKGROUND OF THE INVENTION

Microfluidic device technologies, also referred to as lab-on-a-chip technologies, have been proposed for a number of different applications in various fields. In the field of biology, for example, microfluidic devices may be used to carry out cellular assays. In addition, microfluidic devices have been proposed to carry out separation techniques in the field of analytical chemistry. Microfluidic technology is used in systems that perform chemical and biological analysis, as well as chemical synthesis, on a much smaller scale than previous laboratory equipment and techniques. Microfluidic systems offer the advantages of only requiring a small sample of analyte or reagent for analysis or synthesis, and dispensing a smaller amount of waste materials. A typical microfluidic channel or region of a microfluidic system has at least one cross-sectional dimension in the range of approximately 0.1 micrometers to 1000 micrometers. Since microfluidic technologies involve the use of small volumes of fluids, microfluidic technologies are particularly desirable in applications that involve fluids that are extremely rare and/or expensive. One useful chromatographic function served by microfluidic devices is the separation of proteins.

### DESCRIPTION OF THE DRAWINGS

FIG. 1: is a plan view of an embodiment of a microfluidic reverse affinity-blot device of the present disclosure.

FIG. 2: is an enlarged plan view of area 125 of FIG. 1.

FIG. 3: is a plan view of a second embodiment of a microfluidic reverse affinity-blot device of the present disclosure.

FIG. 4: is a section view of a system including a microfluidic reverse affinity-blot device of the present disclosure.

### DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

Additional advantages and novel features of the invention will be set forth in part in the description that follows, and in part will become apparent to those skilled in the art upon examination of the following, or may be learned through routine experimentation upon practice of the invention.

The microfluidic reverse affinity-blot device of the present disclosure combines affinity binding for isolation and/or enrichment of a selected number of protein analytes from a sample, followed by separation and identification thereof. In general terms, a microfluidic reverse affinity-blot device is a closed system of interconnected components that is comprised of defined points of entry and exit, wherein the interconnected components include a capture region upstream from a protein separation region and subsequent detection region. Different embodiments of the present disclosure will differ in the number of additional interconnected components and defined points of entry and exit. Generally the microfluidic reverse affinity-blot device is designed to hold, channel, or direct fluid or separation media from one location to another with in the interconnected components of the device. Different embodiments will have different numbers of connections and hold, channel and direct fluid or separation media in alternative ways. Generally, the closed system of interconnected components of a microfluidic reverse affinity-blot device is held by a substrate. Typically, the interconnected components of a microfluidic reverse affinity-blot device collectively occupy an area of a substrate less than or equal to about 0.1 cm² to 10 cm².

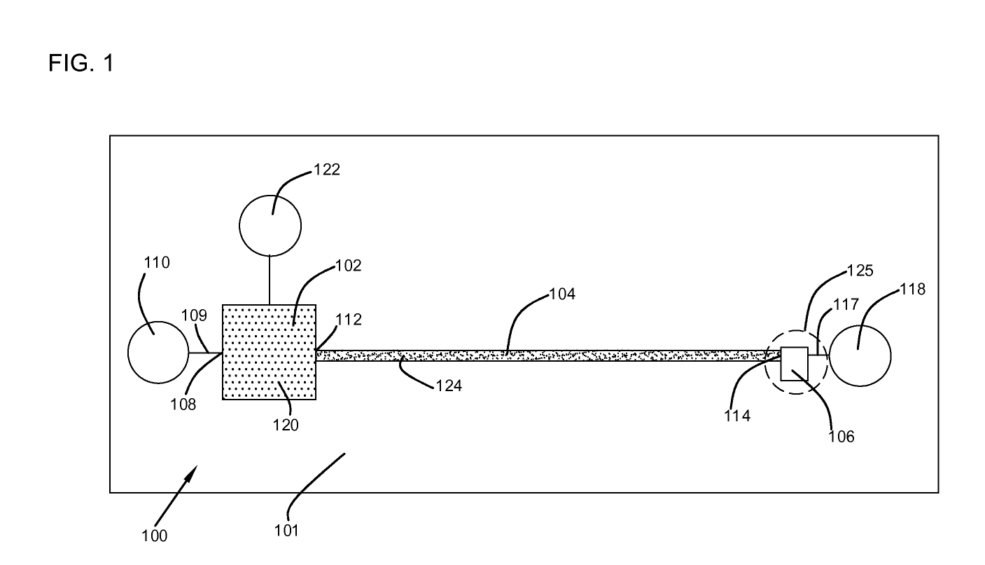

One embodiment of microfluidic reverse affinity-blot device of the present disclosure is described in context of the plan view of FIG. 1. Referring now to FIG. 1, microfluidic reverse affinity-blot device 100 is supported by substrate 101. Substrate 101 defines capture region 102, separation region 104, and detection region 106. Capture region 102 is fluidically connected at first end 108 by microchannel 109 to sample well 110. In addition, Capture region 102 is fluidically connected at second end 112 to protein separation region 104. Protein separation region 104 is additionally fluidically connected to detection region 106 at end 114 opposite capture region 102. Detection region 106 is additionally fluidically connected at opposite end 116 by microchannel 117 to terminal well 118. Additional loading well 122 is fluidically connected by microchannel 121 to third end 123 of capture region 102. In an embodiment, loading well 122 holds binding-disruptive reagent for delivery into capture region 102.

Capture region 102 includes immobilized capture agents 120 held therein. In an embodiment, capture region 102 includes a modified surface or affinity matrix for immobilization of capture agents 120. In an embodiment, capture region 102 includes one or more walls, floor, or ceiling (not shown) of capture region 102 which are modified to bind immobilized capture agents 120 upon contact therewith. In the embodiment of FIG. 1, capture region 102 has at least one modified surface with immobilized capture agents 120 bound thereto.

In a further embodiment, capture region 102 includes an affinity matrix, such as but not limited to, membrane, beads or polymeric matrix wherein the immobilized capture agents bind upon contact therewith. In a still further embodiment, capture region 102 includes affinity matrix bound with immobilized capture agents 120. In a further embodiment, affinity matrix is held within capture region 102 by a barrier (not shown) at end 112 which selectively allows fluid flow-through into separation region 104 including at least some sample components.

Capture agent 120 is a known moiety which has selective binding affinity for a protein analyte in a sample. The term “binds specifically” or “selective binding affinity,” when used in reference to a capture agent, such as an antibody, means that an interaction of the capture agent and a particular epitope on a protein analyte has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. In additional embodiments, immobilized capture agents 120 also have binding affinity to an affinity matrix or modified surface of capture region 104. In one embodiment, the immobilized capture molecules bind irreversibly in capture region 104. In an alternative embodiment, the immobilized capture molecules bind upon contact to capture region 104, but are releasable under certain binding-disruptive conditions.

In various embodiments, immobilized capture agents 120 for use in microfluidic reverse affinity-blot device of the present disclosure include: antibodies, including fragments thereof; affibodies; aptamers; avimers; specific binding partners, including naturally-occurring or derivatives thereof; which have selective binding affinity for a protein analyte in a sample. In various embodiments, capture region 102 includes one or more species of immobilized capture agents 120, wherein each specie of immobilized capture agent 120 has binding specificity for a different protein analyte. In a further embodiment, capture region 102 includes at least one specie of immobilized capture agent 120 which binds selectively to a protein analyte in the sample and at least one other specie of immobilized capture agent 120 which binds selectively to a marker protein. In a still further embodiment, one or more standards (or marker proteins) are added to a sample prior to loading.

As used herein, the term “antibody” is used in its broadest sense to include polyclonal and monoclonal antibodies, as well as antigen binding fragments of such antibodies. An antibody useful in a method of the invention, or an antigen binding fragment thereof, is characterized, for example, by having specific binding activity for an epitope of a protein analyte. In an embodiment, antibodies include naturally occurring antibodies as well as non-naturally occurring antibodies, including, single chain antibodies, chimeric, bifunctional, and humanized antibodies, as well as antigen-binding fragments, such as, Fab, F(ab′)₂, Fd and Fv fragments, that retain specific binding activity for an epitope of a protein analyte. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995)). Monoclonal antibodies suitable for use as probes may also be obtained from a number of commercial sources. Such commercial antibodies are available against a wide variety of targets. Antibody probes can be conjugated to molecular backbones using standard chemistries, as discussed below.

In an embodiment, the immobilized capture agent includes a specific binding partner of a protein analyte. In further embodiments, the specific binding partner is a naturally occurring specific binding partner, such as a ligand or a receptor of the protein analyte. In an alternative further embodiment, the specific binding partner is a synthetic specific-binding partner of the protein analyte (i.e. receptor), or an appropriate derivative of the natural or synthetic ligands of the protein analyte. The determination and isolation of ligands and receptors is well known in the art (Lerner et al. (1994) Trends Neurosci. 17:142-146).

A protein analyte which has binding affinity with an immobilized capture agent 120, and/or a protein analyte which binds to, is bound to, or previously was bound to an immobilized capture agent 120 is also referred to as a captured protein. Generally, a protein analyte is one specie of protein in a sample for analysis by a microfluidic reverse affinity-blot device of the present disclosure. Typically, a sample for analysis by a microfluidic reverse affinity-blot device of the present disclosure includes a plurality of protein species, wherein one of the plurality or a select number of the plurality are protein analytes for analysis by microfluidic reverse affinity-blot device and assay.

In an embodiment, the immobilized capture agent includes affibodies, which are small protein domains selected by combinatorial approached for specific binding to a protein analyte. In an embodiment, affibodies are designed for binding to a protein analyte by randomization of 13 solvent-accessible surface residues of a stable alpha-helical bacterial receptor domain Z, derived from staphylococcal protein A using monovalent phage display. In certain embodiments, affibodies have a secondary structure similar to the native Z domain and have micromolar dissociation constants (Kd) for their respective targets. Further description is available in Nord et al, (1997) Nat Biotechnol., 8:772-7.

In an embodiment, the immobilized capture agent includes Avimers™, which are small, stable proteins that can act like antibodies and bind selectively to different receptors or ligands are available from Medimmune, Inc. and Avidia, Inc.

In an embodiment, the immobilized capture agent includes aptamers, which are oligonucleic acid or peptide molecules that bind a specific protein analyte. In an embodiment, aptamers are usually created by selecting them from a large random sequence pool using combinatorial screening techniques such as phage display or array technologies, but natural aptamers also exist.

In the embodiment of FIG. 1, capture region 102 additionally includes loading well 122 for delivery of binding-disruptive reagent for releasing the captured proteins from immobilized capture agents 120. In various embodiments, binding-disruption reagents include, but are not limited to, protein-compatible solutions, for example, buffered solutions, containing sufficient concentration of detergent, ions, or salts to disrupt protein-capture agent binding interactions. Example binding-disruption reagents include 1% SDS, 4M or higher salt, pH>8 or pH<5 or combination in buffered aqueous solution.

In general, separation region 104 of a microfluidic reverse affinity-blot device of the present disclosure is suited and equipped for separation of different proteins using chromatographic or electrophoretic techniques. In certain embodiments, the proteins are separated based upon one or more of their characteristics, such as, but not limited to size, charge, hydrophobicity, shape, flexibility, oligomeric state, etc. or a combination thereof.

Separation region 104 includes a separation media 124. In various embodiments, separation region 104 is filled with separation media 124, as shown in the embodiment of FIG. 1. Alternatively, a portion of separation region 104 is filled with separation media 124. In still other embodiments, separation media 124 is immobilized on inner surfaces or walls of separation region 104.

In general, separation region 104 of a microfluidic reverse affinity-blot device of the present disclosure is suited and equipped for separation of different proteins using chromatographic or electrophoretic techniques, wherein proteins are separated based upon one or more of their characteristics, such as, but not limited to size, charge, hydrophobicity, shape, flexibility, oligomeric state, etc. or a combination thereof.

In various embodiments, separation media 124 is suitable to effect separation of proteins. In some embodiments, separation media 124 is suitable to effect liquid chromatographic separation of proteins. In embodiments effecting separation of proteins by liquid chromatography, a fluid mobile phase is present in addition to separation media 124. Separation media 124 is generally in continuous contact with fluid mobile phase, and effects separation in conjunction with the mobile phase.

In embodiments where separation media 124 is suitable to effect liquid chromatographic separation of proteins, separation media 124 includes beads and gel matrices. In an embodiment, a separation media is suitable for size exclusion chromatography, gel filtration chromatography, or reverse phase chromatography. In a further embodiment, separation media 124 is a size exclusion or gel filtration media. In still further embodiments, the size exclusion or gel filtration media includes agarose, dextran, polyacrylamide, derivatives thereof, or combinations thereof. Example size exclusion or gel filtration media are available under the tradenames Superose®, Sephadex®, Superdex®, Sephacryl® from Amersham Biosciences, Inc. A variety of other suitable size exclusion, gel filtration, and reversed phase solid media for chromatographic separation of proteins are commercially available.

In other embodiments, separation media 124 is suitable to effect separation of proteins by electrophoresis. Suitable separation media for electrophoretic separations of proteins includes sieving matrices, such as, but not limited to polyacrylamide, agarose, dextran or combinations thereof. In such embodiments, separation media 124 is generally prepared as a gel matrix also including an electrically-conductive buffer solution. A variety of these and other suitable separation media for electrophoresis of proteins are commercially available. In such embodiments, separation region 104 additionally includes electrode connections (not shown).

In one embodiment, separation media 124 for electrophoretic separation is a polyacrylamide gel. An embodiment of the present invention may have a polyacrylamide media including polyacrylamide by weight at between 0.5% and 25%, between 7% and 20%, between 10% and 15%, or about 12% prepared as a gel including an electrically-conductive buffer solution. In an embodiment, the polyacrylamide gel is non-denaturing to proteins. In an alternative embodiment, the polyacrylamide gel is protein denaturing. Denaturing polyacrylamide gels include denaturants, such as detergents, salts, or combinations thereof. In further embodiments, a denaturing polyacrylamide gel includes a concentration of sodium dodecyl sulfate (SDS) or sodium dodecyl phosphate typically between 0.01% to 5%, often between 0.1% to 3%, sometimes between 0.2% to 1%, or occasionally 0.25%.

The separation region 104 of the microfluidic device of the present invention may come in different lengths, widths, and depths, and contain different volumes of separation media. In an embodiment, the dimensions of the separation chamber are selected based upon the volume of separation media required for resolution of typical size range and load of a protein sample for separation in a period of time. In various embodiments, the separation region may be between 17.5 mm and 1.5 mm long, between 15.5 mm and 5.5 mm long, or between 12.5 mm and 8.5 mm long. In various embodiments, the separation region may be between 2 μm and 25 μm deep, between 7 μm and 18 μm deep, between 11 μm and 14 μm deep, or 13 μm deep. In various embodiments, separation region is between 5 μm and 100 μm wide, between 20 μm and 80 μm wide, between 30 μm and 50 μm wide, between 35 μm and 40 μm wide, or 36 μm wide.

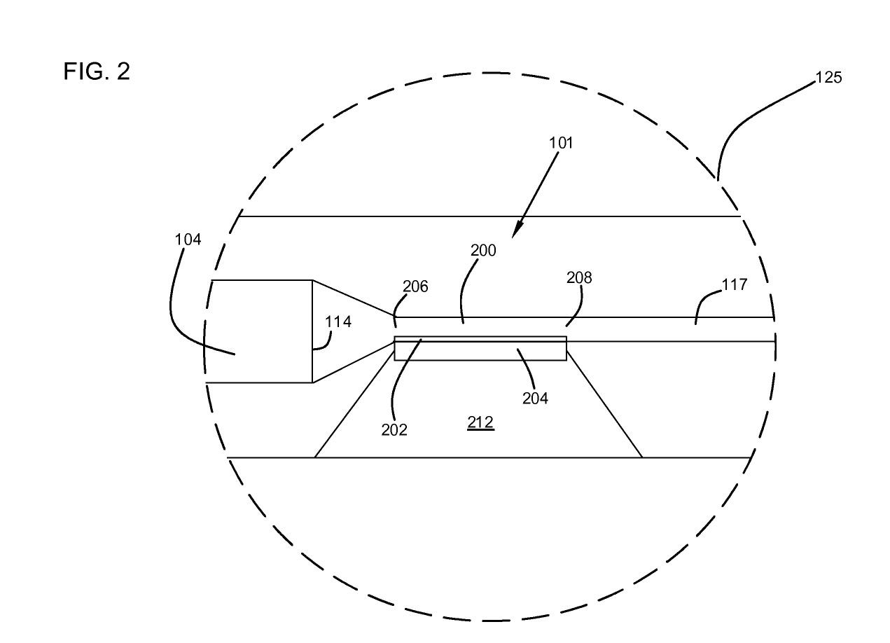

Detection region 106 is generally suited and equipped for detection of proteins. An enlarged cross-section of area 125, including an embodiment of detection region 106 for surface plasmon resonance (SPR) detection, is provided in FIG. 2. In the greatly enlarged embodiment of FIG. 2, detection region 106 includes channel 200, metal surface 202, and transparent illuminating body 204. Channel 200 fluidly connected at first end 206 to end 114 of separation region 104 and is fluidly connected at second end 208 to terminal well 118. Metal surface 202 is positioned on interior surface 210 of channel 200 between first end 206 and second end 208. In an alternative embodiment, metal surface 202 is a metal coating on interior surface 210 of channel 200. In another alternative embodiment, metal surface 202 is a plurality of metal nanoparticles attached to interior surface 210 of channel 200. Transparent illuminating body 204 is positioned adjacent metal surface 202 outside of channel 200. Transparent illuminating body 204, in various embodiments, is a prism or grating. In many embodiments, transparent illuminating body 204 is accessible to an external energy source and sensor.

Metal surface 202, in various embodiments, is a thin layer formed from surface plasmon resonance active metals or combination of surface plasmon resonance active metals. In further embodiments, metal surface 202 is formed from materials including one or more noble metals. In a still further embodiment, the metal surface 202 is formed from metals including gold, silver, platinum, and copper. In other embodiments, the metal surface 202 includes nickel. In still further embodiments, the metal surface 202 includes nickel overlaid with gold or silver.

In alternative embodiments, detection region 106 is adapted for UV/VIS detection. In one such embodiment, metal surface 202 and/or transparent illuminating body 204 are omitted or repositioned suitable for UV/VIS spectroscopic measurement. In such embodiments, channel 200 in substrate 101 is formed of transparent materials.

In the embodiment of FIG. 2 and various other embodiments, opening 212 in substrate 101 is provided for operative connection to an energy source and sensor. In an alternative embodiment, substrate 101, at least in the area designated opening 212, is formed of transparent material for operative connection to an energy source and sensor provided external to microfluidic reverse affinity-blot device. In embodiments wherein substrate 101 or portions thereof are formed of transparent materials, the transparent materials typically emit low fluorescence upon illumination with the light. In further embodiments, highly transparent materials are typically used so as to reduce the absorption of the incident illumination to improve detection of light reflected through the substrate as well as to avoid substrate heating. In such embodiments, substrate 101 or portions thereof will transmit at least 85%, 90%, or 95%, of the illuminating light incident on the substrate as may be measured across the entire integrated spectrum of such illuminating light or alternatively at selected wavelengths of visible light or from infrared or UV light sources. In various embodiments substrate materials include: glass, quartz, silica and/or polymeric materials. Polymeric materials include polyimide, polycarbonate, polystyrene, polyolefin, polyvinyl fluoride, polyester, a nonaromatic hydrocarbon, polyvinylidene chloride, polyhalocarbon.

In one embodiment of the microfluidic reverse affinity-blot device of FIGS. 1 and 2, the interoperability of the regions is described as follows. In most embodiments, movement of sample and reagents within microfluidic reverse affinity-blot device is mediated by fluid movement and/or pressure. Fluidically connected sample well 110, microchannel 109, capture region 102, protein separation region 104, detection region 106, microchannel 117, and terminal well 118 provides for controlled flow of sample, portions thereof, reagents, and other fluids (not shown) flow from sample well 110 into and through the fluidically connected regions and into terminal well 118. In an embodiment, a sample having a protein analyte, generally in a buffered solution, is loaded in sample well 110. During operation of the microfluidic reverse affinity-blot device 100 of FIG. 1, as described further below, sample moves from sample well 110 into capture region 102, wherein immobilized capture agents 120 specifically bind to typically at least a portion of the sample.

In an embodiment according to FIG. 1, unbound and/or non-specifically bound sample, if present, moves out of capture region 102, typically by action of additional buffered solution provided from sample well 110 under fluid pressure. In alternative embodiments, additional buffered solution is provided from another reagent supply well (not shown) fluidically connected to either sample well 110 or capture region 102. In the embodiment of FIG. 1, the unbound and/or non-specifically bound sample moves into and separates while passing through protein separation region 104. The separated unbound and/or non-specifically bound sample moves out of the protein separation region 104 into detection region 106, where it is detected, and subsequently moves into terminal well 118.

After unbound and/or non-specifically bound sample moves out of capture region 102, binding-disruptive reagent (not shown) moves from loading well 122 into capture region 102 for release of protein analytes from the sample (i.e. “captured proteins”) from binding interactions with the capture agents 120. The captured proteins are moved into separation region 104. In separation region 104, the captured proteins contact separation media 124 and are separated while passing through. In one embodiment, movement of captured proteins through separation region 104 and separation media 124 is via application of electrophoretic force. In another embodiment, mobile phase (not shown) for separation of capture proteins is provided from loading well 122. In alternative embodiments, a mobile phase supply well connected to end 112 of separation region 104 is provided.

Referring now to FIG. 2, separated captured proteins move out of protein separation region 104 into channel 200 of detection region 106. In an embodiment, separated captured proteins move out of protein separation region 104 carried by mobile phase. In channel 200, the separated captured proteins interact with metal surface 202, are detected, and subsequently move through channel 200 into terminal well 118.

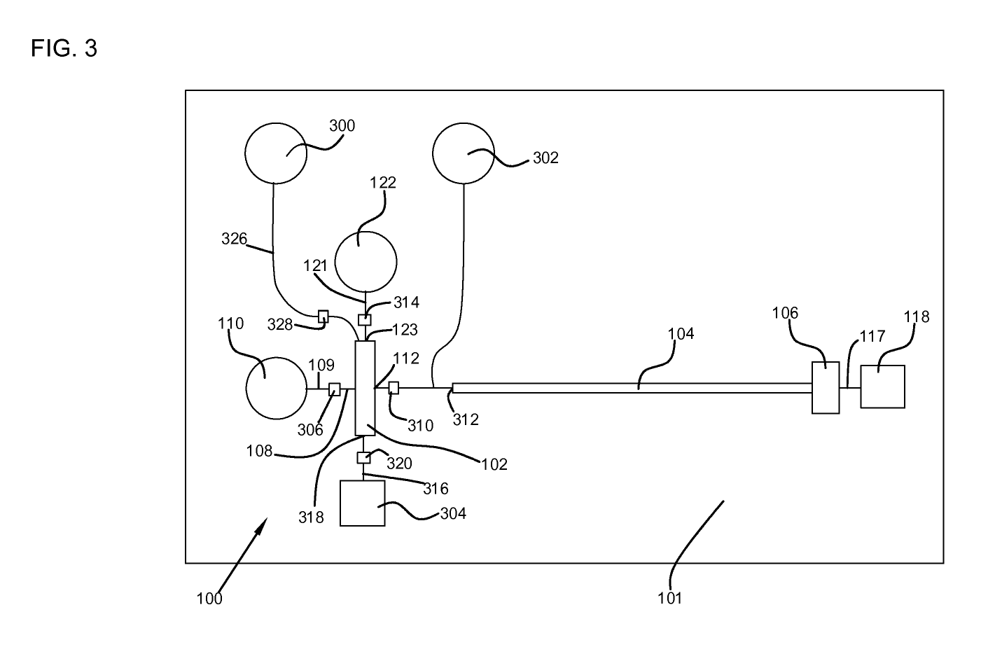

Additional embodiments of microfluidic reverse affinity-blot device 100 differ in the number of additional interconnected components and defined points of entry and exit from the embodiments presented in FIGS. 1 and 2. Further additional embodiments include, for example, additional reagent supply wells and terminal or waste wells. Other embodiments include additional interconnected components to facilitate, control and direct fluidic connection. Further embodiments include one or more valves and optionally further microfluidic channels, for example, but not limited to as shown in FIG. 3.

Another embodiment of microfluidic reverse affinity-blot device 100 is illustrated in the plan view of FIG. 3. FIG. 3 includes substrate 101, capture region 102, capture agent 120, separation region 104, detection region 106, sample well 110, terminal well 118, loading well 122, and other components as generally described for FIG. 1. In addition, the embodiment of FIG. 3 includes additional loading wells 300, 302, and terminal well 304. FIG. 3 additionally illustrates selected regions and wells of the microfluidic reverse affinity-blot device 100 fluidically connected by microfluidic channels 109, 117, 121, 308, 316, 322, 326 and valves 306, 310, 314, 320, 324, 328. Microfluidic channel 109, with valve 306 positioned therein, fluidically connects sample well 110 with capture region 102. Capture region 102 is fluidically connected at second end 112 with microfluidic channel 308. Valve 310 is positioned in microfluidic channel 308, which is fluidically connected to first end 312 of protein separation region 104. Protein separation region 104 is additionally fluidically connected at opposite end 114 to detection region 106. Detection region 106 is additionally fluidically connected at opposite end 116 to terminal well 118 via microfluidic channel 117. In one embodiment of FIG. 3, a means for releasing the capture proteins includes loading well 122, which is fluidly connected to microfluidic channel 121, which is further fluidically connected to third end 123 of capture region 102. Microfluidic channel 121 has valve 314 positioned therein for control of binding-disruptive reagent (not shown) delivery. In addition, FIG. 3 provides additional terminal well 304 fluidically connected to microfluidic channel 316 which is fluidly connected to a fourth end 318 of capture region 102. Microfluidic channel 316 includes valve 320 positioned therein.

Interoperability of an embodiment of FIG. 3 is described as follows. In an embodiment, microfluidic reverse affinity-blot device 100 is primed with buffer or other fluid, and has been preloaded with capture agents 120 and separation media 124. In various embodiments, additional reagents and components are loaded into additional loading wells, 122, 300 and/or 302. In further embodiments, capture agents 120 and/or separation media 124 are loaded into microfluidic reverse affinity-blot device 100 via loading wells 122, 300, and/or 302.

A sample, in a buffered solution, suspected to have one or a select number of protein analytes, is loaded in sample well 110. First, valve 306 opens allowing sample to move from sample well 110, into microfluidic channel 109, through valve 306 and into capture region 102, wherein immobilized capture agents 120 specifically bind to one or a select number of protein analytes, if present. In various embodiments, at least one valve of 310 or 320 is also open to allow displacement of fluid from capture region 102. In further embodiments, valves 310, 314, are closed, while valve 320 is open to allow fluid to be displaced from the capture region 102 into terminal well 304. In other further embodiments, valves 310, 320, are closed, while valve 314 is open to allow fluid to be displaced from the capture region 102 into microfluidic channel 308.

In some embodiments, valve 306 is closed when sample occupies capture region 102, for example when a volume of fluid approximately equal to the capture region 102 is displaced by sample moving into capture region 102. In a yet further embodiment, valves 306, 310, 314, and 320 are closed to contain sample in capture region 102 for a period of time. In an embodiment, a period of time is equal to or in excess of time for binding equilibrium to be reached for protein analyte to capture agent 120 binding.

In an embodiment according to FIG. 3, unbound and/or non-specifically bound sample, if present, moves out of capture region 102, typically by fluid pressure from buffered solution provided by sample well 110. In alternative embodiments, additional buffered solution is provided from another reagent supply well (not shown) fluidically connected to either sample well 110 or capture region 102. In one further embodiment, valves 306, 310, 314 are closed, while valve 320 is open for movement of unbound and/or non-specifically bound sample into terminal well 152.

After unbound and/or non-specifically bound sample moves out of capture region 102, valve 314 opens for movement of binding-disruptive reagent (not shown) from loading well 122 via microfluidic channel 130 into capture region 102 for release of captured proteins (not shown) from binding interactions with the capture agents 120. In a further embodiment, valve 306 is closed, while valve 310 and/or 320 is open to allow fluid to be displaced from the capture region 102. In a still further embodiment, valves 306, 310, 320 are closed when binding-disruptive reagent occupies capture region 102, for example when a volume of fluid approximately equal to the capture region 102 is displaced by binding-disruptive reagent moving into capture region 102. In a yet further embodiment, valves 306, 310, 314, 320 are closed to contain binding-disruptive reagent in capture region 102 for a period of time. In an embodiment, a period of time is equal to or in excess of time needed for disassociation equilibrium to be reached for protein analyte dissociation from capture agent 120.

Next, valve 310 is opened for movement of captured proteins, now released from capture agent 120, separation region 104 via microfluidic channel 308. In one embodiment of separation region 104, the captured proteins contact separation media 124 and are electrophoretically separated while passing through. In another embodiment of separation region 104, the captured proteins contact separation media 124 and are liquid chromatographically separated while passing through. In one embodiment of separation region 104 for liquid chromatographic separation, one or more additional wells 302 are fluidically connected by one or more additional microchannels 322 and valves 324 to separation region 104 to provide mobile phase (not shown). In other embodiments, additional wells 302 are provided for loading of separation media 124.

In both of the above embodiments, separated captured proteins (not shown) move out of protein separation region 104, into detection region 106, where they are detected, and subsequently move into terminal well 118.

Additional embodiments of FIG. 3 differ in the number of additional interconnected components and defined points of entry and exit. Further additional embodiments include, for example, additional reagent supply wells and terminal or waste wells. Other embodiments of FIG. 3 include additional interconnected components and/or microfluidic channels to facilitate, control and direct fluidic connection. Further embodiments of FIG. 3 include other valve configurations including more or less valves and/or alternative placement relative to regions and wells to operably separate regions, channels, and wells and/or to control direction and timing of movement of reagents, sample and portions thereof, into and through components of microfluidic reverse affinity-blot device 100. Additional embodiments of FIG. 3 differ in operation, for example in sequence and timing of opening and closing of one or more of valves 306, 310, 314, 320, 324, 328.

### Substrate

In some embodiments, the substrate upon which microfluidic features are disposed or defined is a substrate known in the art to be suitable for the fabrication of microfluidic devices. Examples of suitable substrates include glass, silicon, plastic, or combinations thereof. In some embodiments, polydimethylsiloxane (PDMS) is the substrate. Some advantages of PDMS are that it is inexpensive, optically clear, and permeable to several substances, including gases. Since air can quickly diffuse out, the latter aspect is very convenient, making it possible to inject fluid into a channel that has no outlet. In other embodiments, poly(ether ether ketone) (PEEK) is the substrate. Advantages associated with PEEK include excellent mechanical properties and resistance to thermal degradation. Glass and polyimide are other commonly used substrate materials in microfluidic applications.

In some embodiments, substrate 101 of microfluidic reverse affinity-blot device 100 defines fluid connections between features that are microfluidic channels. Typically, in embodiments, channels connecting various features on the device have a width of about 10 micrometers to about 100 micrometers and a depth of about 5 micrometers to about 50 micrometers.

In some embodiments, microfluidic reverse affinity-blot device 100 includes one or more valves. The valves are, in embodiments, mechanical valves such as those described in U.S. Pat. No. 6,702,256, which is incorporated herein by reference in its entirety. In other embodiments, the microfluidic reverse affinity-blot device has valves including a thermoelectric device capable of providing either addition of heat or removal of heat, and a material capable of phase change upon addition of heat or removal of heat by the thermoelectric device. Valves employing thermoelectric devices and phase changing materials are described in U.S. Pat. Nos. 6,007,302 and 5,975,856, which are incorporated herein by reference in their entirety.

In various embodiments, the microfluidic reverse affinity-blot device includes additional wells, valves, channels, and access points as needed for loading and supply of samples and reagents, control of flow and collection of samples and regents, including waste materials, and degassing.

### Reagents

In some embodiments, the microfluidic reverse affinity-blot device has one or more sources of reagents. Reagents are, in embodiments, capture agents 120, reagents suitable for delivery or immobilization of capture agents 120 into capture region 102, disruption of capture agent-protein interactions, standards, protein denaturation, protein separation, protein detection, or combinations thereof. Thus, the reagents include, in some embodiments, one or more of a buffer solution, and a set of standard proteins or other molecular weight standards. The reagents include, in some other embodiments, one or more of a buffer solution, denaturants, detergents, chromophores or dyes, and a set of standard proteins or other molecular weight standards.

In some embodiments the reagents are preloaded onto the device prior to addition of a protein sample. Generally, a microfluidic reverse affinity-blot device is primed with buffer prior to sample loading. In one such embodiment, one or more types of capture agents 120 are preloaded into capture region 102. In another such embodiment, separation media is preloaded into separation region 104. In various embodiments, capture agents 120 immobilized into capture region 102 are protected from adverse conditions, such as drying and heat above room temperature, prior to use of the microfluidic reverse affinity-blot device. In various embodiments, capture region agents 120 preloaded into capture region 102 is maintained in contact with buffer or mobile phase until use. In various embodiments, separation media preloaded into separation region 104 is maintained in contact with buffer or mobile phase until use.

In some embodiments, one or more of the reagents are present in one or more wells. The wells are disposed on the microfluidic reverse affinity-blot device in addition to sample well 110. In some embodiments, the additional wells are in fluid connection sample well 110. In other embodiments the wells are in fluid communication one or more other components, for example, region, well, or microfluidic channel of the microfluidic reverse affinity-blot device. In a further embodiment, one or more additional wells are in fluid communication with first end 112 of separation region 104 for delivery of one or more regents or mobile phase for separation.

The choice of buffer employed with the microfluidic reverse affinity-blot device is not particularly limited and should be chosen based on the sample proteins being assayed. Examples of buffers commonly employed in embodiments of the present disclosure are tris-HCl, tris-tricine, bis-tris, hepes, mops, TBE (tris borate EDTA), tris acetate EDTA (TAE), urea, glycine, or mixtures of these at various values of pH.

In embodiments, a buffer is used to transport one or more detergents, one or more standard proteins, or one or more other reagents within the microfluidic reverse affinity-blot device. In various embodiments, samples and/or reagents the solid media and/or liquid phase includes a suitable buffer maintaining suitable and/or consistent pH within one or more components, for example regions and/or wells, of the microfluidic reverse affinity-blot device. In various embodiments, a buffer is used to carry sample. In a further embodiment, solid media and/or liquid phase includes a suitable buffer maintaining suitable and/or consistent pH across the separation region.

In various embodiments, reagents, such as buffers, mobile phases, binding-disruption agents, and/or sample, include a detergent. Typically, a detergent is provided in an aqueous solution, typically a buffered aqueous solution. The choice of detergent is not particularly limiting to the various embodiments disclosed herein. In some embodiments, sodium dodecyl sulfate or sodium dodecyl phosphate is the detergent. However, in other embodiments, other anionic surfactants, nonionic surfactants such as, for example, Triton® X-100 ((C₁₄H₂₂O(C₂H₄O)ₙ)) or Tween®-20 (polyoxyethylene sorbitan monolaurate) (available from the Sigma-Aldrich Company of St Louis, Mo.), or zwitterionic detergents such as, for example, 3-[(3-cholamidopropyl) dimethylammonio]-1-propanesulfonate (CHAPS), are used as the detergent.

In various embodiments, a mobile phase is provided into at least the separation region 104, as described above. In some embodiments, a mobile phase is an aqueous buffer solution. In other embodiments, a mobile phase is an isocratic or gradient mobile phase. In further embodiments, isocratic or gradient mobile phases additionally include organic solvents in combination with aqueous buffers. In still further embodiments, additional additives are included to facilitate protein separation. An example mobile phase for protein separation is acetonitrile in aqueous trifluoroacetic acid.

### Standards

In various embodiments, a set of standard proteins is analyzed in addition to, typically concurrently with protein analyte in a sample. In an embodiment, as set of standard proteins is present in a well that is in fluid connection to capture region 102. In a further embodiment, capture region 102 includes one or species of capture agents 122 for capture of standard proteins. In another embodiment, a set of standard proteins is present in a well that is in fluid connection to separation region 104, such that the standard proteins are eluted through separation region 104. The standard proteins, in some embodiments, are present in a buffer solution. In some embodiments, standard proteins are added to the sample or to sample loading well 110. Standard proteins that are employed in some embodiments include one or more of bovine serum albumin, albumin, cytochrome C, myoglobin, carbonic anhydrase I, carbonic anhydrase II, lysozyme, Beta-lactoglobin, soybean trypsin inhibitor, ovalbumin, and Beta-galactosidase. However, in other embodiments different standard proteins of combinations thereof are used. The choice of standard proteins is not particularly limiting to the various embodiments disclosed herein. In certain embodiments, standard proteins are employed in the microfluidic reverse affinity-blot device as a co-elution standard of known molecular weight, against which an unknown protein sample can be measured for an accurate comparative determination of molecular weight. In other embodiments, standard proteins are employed in the microfluidic reverse affinity-blot device for either or both positive or negative controls for affinity binding in capture region 102. Standard proteins may be pre-labeled with fluorescent dyes or other detection molecules or they may be detected in the same manner as the proteins in the sample.

In some embodiments, chromophores and fluorescent dyes are used as internal standard for determination of molecular weight. For example, in embodiments, chromophores or small molecules are selected based on their molecular weight and mass-to-charge ratio. These markers are validated against various protein standards of known molecular weight and mobility in the separation assay. Small molecular weight standards can have the advantages of stability over protein standards, and thus may increase the consistency and ease of use of the assay.

In some embodiments, a detection agent such as a dye, fluorescent molecule or other detection label is used to aid in detection of the sample, including captured proteins, and/or standard proteins. Non-limiting examples of dyes include Coomassie® Brilliant Blue or SYPRO® Ruby, available from Invitrogen Corporation of Carlsbad, Calif., USA or Krypton™ protein stain available from Pierce Biotechnology, Rockford, Ill., USA. Non-limiting examples of fluorescent labeling molecules include NHS-Fluorescein, NHS-Rhodamine, or the Dylight™ protein labeling dyes available from Pierce Biotechnology, Rockford, Ill., USA. Examples of other detection labels include quantum dots available from Invitrogen Corporation, Carlsbad, Calif., USA. The detection agent may be covalently or noncovalently associated with the proteins in the sample or the standards. Suitable protein detection agents and methodologies are employed in conjunction with these embodiments.

### System

In various embodiments, the microfluidic device of the present disclosure is designed to be inserted into or assisted by an instrument, for example, the Agilent 2100 Bioanalyzer by Agilent Technologies, Inc. in Santa Clara, Calif. One skilled in the art knows the Agilent 2100 bioanalyzer or similar instruments. The necessary requirements of the instrument will be understood by one of ordinary skill in the art. In embodiments where the microfluidic reverse affinity-blot device is designed to be inserted into or assisted by an instrument, the instrument typically provides one or more of the following: heat source, detector source and a computer monitor for displaying the results of detection, pumps to provide pressure, power sources, electrodes or electrode contacts for electrophoresis.

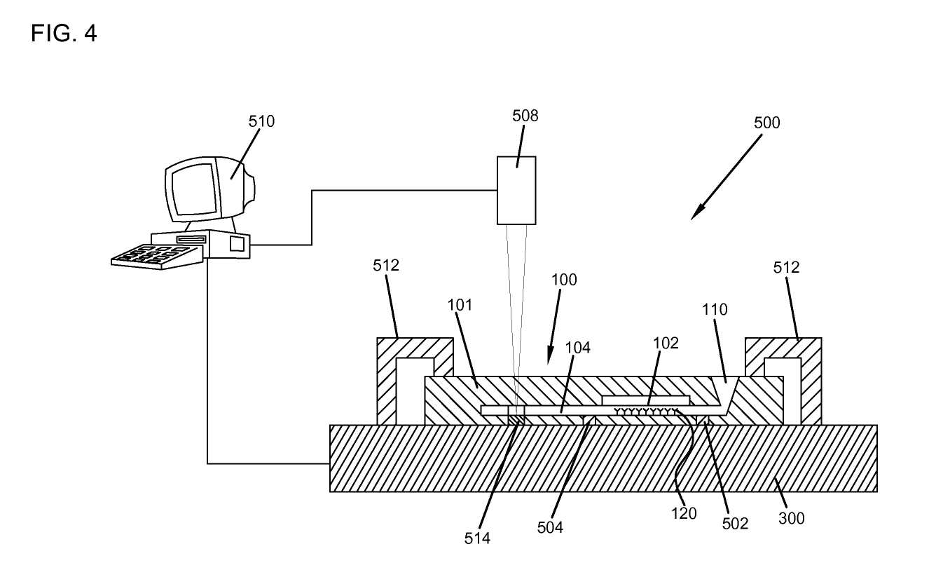

Referring to FIG. 4, a system 500 is shown. System 500 incorporates microfluidic reverse affinity-blot device 100 having substrate 101 defining features. Defined in substrate 101 are features including well 110, capture region 102, separation region 104, first valve 502 disposed between well 110 and capture region 102, and second valve 504 disposed between capture region 102 and separation region 150. Valves 162, 164 comprise thermoelectric elements. System 500 further includes an optical interrogation module 508, which analyzes a protein analytes as they progress through separation region 104. In some embodiments, system 500 includes an external computer 510. For example, external computer 510 can be in data connection with at least optical interrogation module 508. System 500 further includes clamps 512 to provide a secure connection for microfluidic reverse affinity-blot device 100 to additional instrument portions 300 of system 500.

In some embodiments, external computer 510 is present as part of an instrument in data communication with one or more features of microfluidic reverse affinity-blot device 100 or system 500, for example with valves 502, 504, and optical module 508.

In some embodiments of a system 500 for reverse affinity blot, a sample including protein analyte moves through the separation region 104 of the microfluidic reverse affinity-blot device 100 by pressure, electrophoresis, or a combination thereof. In some embodiments, the pressure and electrophoresis are supplied to the separation region 104 of the microfluidic reverse affinity-blot device 100 by an instrument in which the microfluidic reverse affinity-blot device is inserted, for example, the Agilent 2100 Bioanalyzer® by Agilent Technologies, Inc. of Santa Clara, Calif. In some embodiments, the instrument provides both pressure and electrophoretic mobility, individually or simultaneously, to the separation region 104 of the microfluidic reverse affinity-blot device 100. In some embodiments, external computer 510 is in data communication with one or more separation regions 104, one or more sources of pressure or electrophoretic mobility, or any combination thereof. In embodiments, external computer 510 interprets data signals from separation region 104. In other embodiments, external computer 510 provides signals to one or more sources of pressure or electrophoretic mobility in order to control movement of a sample through one or more separation regions 104.

In some embodiments of a system 500 including microfluidic reverse affinity-blot device 100, system 500 includes a detector source, such as an optical interrogation module 508. Optical interrogation module 508 can provide an optical signal to separated capture proteins sample eluting through separation region 104. In embodiments, the interaction of the optical signal with the eluting separated captured proteins provides data for analysis of the sample. For example, the result of the interaction of the optical signal and the eluting sample may be a modified signal, which is detected by a detection apparatus 514. In embodiments, a detected signal is sent to external computer 510, which interprets the modified signal and presents results in readable form for a human user. In some embodiments, the optical interrogation module 508 provides a signal that interacts with a sample to provide an ultraviolet measurement, a fluorescence measurement, or a surface plasmon resonance measurement.

### Methods

In embodiments, in a method of performing a reverse affinity-blot assay on a microfluidic reverse affinity-blot device 100 is provided. In an embodiment, a reverse affinity-blot assay is described in the context of microfluidic reverse affinity-blot device 100 according FIG. 3.

In various embodiments, microfluidic reverse affinity-blot device 100 is primed prior to use. Priming of the system includes loading of the microfluidic reverse affinity-blot device with liquid, such as a buffered aqueous solution for the exclusion of air or other gases from the microfluidic reverse affinity-blot device.

In some embodiments, the method further involves addition of at least one source of reagents to the microfluidic reverse affinity-blot device 100. In various embodiments, reagent solutions and sample required by the microfluidic reverse affinity-blot device for assay are loaded into designated wells. The reagents include, in embodiments, a buffer, an assay standard protein, capture agents 120, separation media 124, and mobile phase. In one embodiment of the method, capture agents 122 in a buffered solution are loaded into well 300 fluidly connected via microfluidic channel 326 and controlled by valve 328. In other embodiments, a microfluidic reverse affinity-blot device 100 of the present disclosure is provided preloaded with capture agents 122 immobilized in capture region 102. In one embodiment of the method, separation media 124 in mobile phase or buffered solution is are loaded into loading well 302 fluidly connected via microfluidic channel 322 and controlled by valve 324 into separation chamber 104. In other embodiments, a microfluidic reverse affinity-blot device 100 of the present disclosure is provided preloaded with separation media 124 in separation region 104. In certain embodiments, microfluidic reverse affinity-blot device 100 is preloaded with other reagents necessary to perform the reverse affinity-blot assay. In certain other embodiments, microfluidic reverse affinity-blot device 100 is loaded with reagents necessary to perform the reverse affinity-blot assay by the user.

In some embodiments, the method involves applying about 1 picoliter to 10 microliters of protein sample to the microfluidic reverse affinity-blot device 100.

Next, the loaded microfluidic reverse affinity-blot device 100 is inserted into a system and the system is activated. In an embodiment, the system is for example, the Agilent 2100 Bioanalyzer by Agilent Technologies, Inc. in Santa Clara, Calif. In many embodiments of the reverse affinity-blot assay performed using microfluidic reverse affinity-blot device 100 are automated by a system. In such embodiments, pumping of sample and reagents from loading wells, and opening/closing of valves is controlled by the system.

In certain embodiments, preparation of microfluidic reverse affinity-blot device 100 for assay is needed. In various embodiments, capture agents 120 are directed into capture region 102. In further embodiments, unimmobilized capture agents are removed, for example by buffer wash-through into terminal well 304. In various embodiments, separation media 124 is directed into separation region 104.

Next, the reverse affinity-blot assay begins by pumping protein sample from sample loading well 110, through open valve 306 into capture region 102. Valve 310 and 314 are closed. Unbound sample is removed by pumping of buffer, for example from additional well 300, through capture region 102 into terminal well 304. Next, valves 306 and 320 are closed, while valve 314 is opened and binding-disruptive agent pumped from loading well 122 into capture region 102. In certain further embodiments, binding-disruptive agent from loading well 122 and/or capture region 102 are warmed above room temperature to assist release of captured proteins. Released captured proteins are carried by binding-disruptive agent from loading well 122, buffer from loading well 300, and/or buffer/mobile phase from loading well 302 into separation region 104. In certain embodiments, one or more of loading wells, 122, 300 and 302 continue to provide mobile phase for separation of captured protein as it is moved by the mobile phase through separation media 124. In certain other embodiments, capture proteins are moved into contact with separation media 124, whereby electrophoretic forces assume control of the captured proteins, moving and separating them through the separation media 124, until detection region 106 is reached. Suitable chromatographic or electrophoretic methods include size exclusion chromatography, gel filtration chromatography, reverse phase chromatography, gel electrophoresis, or capillary electrophoresis.

In various embodiments, sample moves through separation region 104 by pressure or electrophoresis or both. In a further embodiment, the pressure and electrophoresis are supplied to the microfluidic device of the present disclosure by an instrument in which the microfluidic reverse affinity-blot device is inserted. In an embodiment, the system is for example, the Agilent 2100 Bioanalyzer by Agilent Technologies, Inc. in Santa Clara, Calif. In embodiments, wherein the microfluidic reverse affinity-blot device is the Agilent 2100 Bioanalyzer can provide both pressure and electrophoretic mobility, individually or simultaneously, to the microfluidic device of the present invention.

In certain embodiments, detection region 106 includes real-time detection of captured proteins as they move through the region. At a point before or after exiting separation region 104, the captured proteins move past a detector. In some embodiments, the method further involves analyzing and/or identifying the separated captured proteins. In some embodiments, the analyzing is accomplished optically, for example, by ultraviolet measurement, fluorescence measurement, measurement of surface plasmon resonance. Fluorescence measurements are carried out by, for example, loading a fluorescent tag bearing chemical as reagent within a well on the microfluidic reverse affinity-blot device 100, such that the tag can become associated with the protein. Such associations allow quantification of fluorescence on a protein molecule that in turn allows quantification of molecular weight of individual species by measuring the level of fluorescent emissions of a species as it is eluted on the separation column. In many embodiments, components of a detector, such as energy source and sensor are provided and controlled by a system holding a microfluidic reverse affinity-blot device 100.

In certain embodiments, surface plasmon resonance is measured in detection region 106. In a further embodiment, referring back to FIG. 2, captured proteins adsorb to a metal surface 202 in detection region 106 while a system applies energy to detection region 106 through transparent illuminating body 204 and measures surface plasmon resonance. In a still further embodiment, interaction of captured proteins with metal surface 202 produces detectable changes in the local index of refraction. In other and additional embodiments for measurement of surface plasmon resonance, metal surface 202 is patterned with affinity molecules for the capture proteins, including but not limited to the capture agents 120 described supra. In certain embodiments where metal surface 202 includes metal nanoparticles, ultraviolet-Visible absorption bands, sometimes including intense colors, are detectable.

### Formation of Microfluidic Reverse Affinity-Blot Device

Microfluidic channels, separation regions, wells, and other microfluidic features are not limited by the technique with which they are formed. Microfluidic channels can be made by any known technique including lithography, laser etching, printing, and microreplication of inverse images of features disposed on PDMS, nickel, or polyimide onto a final substrate such as a thermoplastic. In some embodiments, microreplication is carried out by a melt technique, such as applying heat and pressure to a thermoplastic disposed against a mold having an inverse pattern. In other embodiments, microreplication can be carried out by casting an uncured polymer such as PDMS onto a mold and curing the polymer, followed by removal of the mold. In other embodiments, glass is patterned by photolithography before channels are wet or dry etched. Any of these techniques, or others, can be used to form microfluidic channels on the substrate of the microfluidic reverse affinity-blot device. The described techniques are merely illustrative and do not limit the techniques that can be used to make microfluidic channels.

The various embodiments described above are provided by way of illustration only and should not be construed to limit the claims attached hereto. Various modifications and changes may be made without following the exemplary embodiments and applications illustrated and described herein, and without departing from the scope of the following claims.

The present invention may suitably comprise, consist of, or consist essentially of, any of the disclosed or recited elements. Thus, the invention illustratively disclosed herein can be suitably practiced in the absence of any element which is not specifically disclosed herein.

We claim:

1. A microfluidic device for isolation and/or size determination of one or more proteins in a sample, the microfluidic device comprising:
a loading well for holding a sample for delivery into the microfluidic device;
a capture region fluidically connected to the loading well, wherein the capture region comprises an immobilized capture agent for capture of one or more protein species in the sample, and an elution well for holding binding-disruptive reagent for subsequently releasing the captured proteins;
a protein separation region fluidly connected to the capture region to receive the captured proteins, wherein the protein separation region comprises a separation media for separation of the captured proteins; and
a detection region operatively connected to the protein separation region that detects the captured proteins in the sample after separation.

2. The microfluidic device of claim 1, wherein the capture agent comprises antibodies, aptamers, affibodies, avimers or peptides.

3. The microfluidic device of claim 1, wherein the binding-disruptive reagent comprises detergents, salts, acid, base, or combinations thereof in an aqueous buffered solution, in amounts sufficient to release the captured proteins from the immobilized capture agent.

4. The microfluidic device of claim 1, comprising two or more loading wells, wherein each loading well is individually fluidically coupled to the capture region.

5. The microfluidic device of claim 4, wherein at least one of the loading wells is a sample loading well, and at least one other of the loading wells is a capture agent loading well for delivery to the capture region.

6. The microfluidic device of claim 1, further comprising a collection chamber fluidly connected to the capture region.

7. The microfluidic device of claim 1, wherein the capture region comprises beads, a functionalized surface, a membrane, a matrix, a monolith, or combination thereof for immobilization of capture agents.

8. The microfluidic device of claim 1, wherein the capture region comprises immobilized capture agents of two or more capture agent species having different binding specificities for one or more proteins in a sample.

9. The microfluidic device of claim 1, wherein the separation region comprises a sieving matrix.

10. The microfluidic device of claim 9, wherein the sieving matrix comprises polyacrylamide, agarose, dextran, silica, or a combination thereof.

11. The microfluidic device of claim 10, wherein the sieving matrix resolves proteins from 1 kDa to 3000 kDa.

12. The microfluidic device of claim 1, additionally comprising a mobile phase loading well fluidly connected to the protein separation region.

13. The microfluidic device of claim 1, where in the detection region comprises an energy source and a sensor for detection of surface plasmon resonance, fluorescence, UV/VIS absorption, or a combination thereof of one or more proteins in a sample.

14. The microfluidic device of claim 1, wherein detection region comprises a channel, pathway, lens, window or combination thereof for the operative connection of an external energy source to the separation region.

15. The microfluidic device of claim 1, where in the detection region comprises
a metal surface or metal nanoparticles, wherein the metal surface or metal nanoparticles interact with at least one protein in the sample; and
a transparent illuminating body adjacent the metal surface or metal nanoparticles.

16. A method of isolating and identifying a protein in a sample using the microfluidic device of claim 1, the method comprising:
applying the sample to the loading well of the microfluidic device of claim 1;
capturing a protein on the immobilized capture agent;
eluting the captured protein from the immobilized capture agent to the protein separation region;
separating captured protein in the protein separation region; and
detecting separated protein.

17. The method of claim 16, wherein the eluting comprises applying eluent to the immobilized capture agent wherein the eluent is characterized by high pH, low pH, high salt, eluent comprising detergent, or combinations thereof.

18. The method of claim 16, wherein the immobilized capture agent is an antibody.

19. The method of claim 16, wherein separating captured protein in the protein separation region comprises moving capture protein through separation media by either applying mobile phase or by applying charge.

20. The method of claim 16, wherein the detecting comprises
adsorbing the separated proteins to a metal surface;
applying energy to the metal surface; and
detecting the energy reflected away from the metal surface.

21. The method of claim 16, wherein the detecting additionally comprises:
labeling said protein in the sample with a detection label;
applying energy to the labeled separated protein; and
detecting the energy generated by the labeled separated protein.

22. A method of isolating and identifying protein of interest in a sample, the method comprising:
immobilizing capture agents on a solid support, wherein the immobilized capture agents have specific binding affinity to said protein of interest;
contacting said solid support with the sample;
binding said protein of interest to said immobilized capture agent;
washing said solid support to remove sample not bound to said immobilized capture agent;
eluting captured protein from said immobilized capture agent;
applying said eluted protein to a separation device;
separating said eluted protein on said separation device; and
detecting said separated protein.
